(19) United States
(12) Patent Application Publication
(10) Pub. No.: US 2026/0232977 A1
(43) Pub. Date: Aug. 13, 2026

(54) MICRONEEDLE FOR INTRADERMAL FILLING

(71) Applicants: Avraham AMIR, Tel-Mond (IL); Mark SHAHAR, Tel-Aviv (IL)

(72) Inventors: Avraham AMIR, Tel-Mond (IL); Mark SHAHAR, Tel-Aviv (IL); Nir BARKAI, Kfar-Sava (IL)

(21) Appl. No.: 19/157,462

(22) PCT Filed: Feb. 15, 2024
(86) PCT No.: PCT/IL2024/050177

§ 371 Date: Aug. 18, 2025

Related U.S. Application Data
(60) Provisional application No. 63/446,390, filed on Feb. 17, 2023.

(51) Int. Cl. A61M 37/00; A61M 5/32; A61M 5/46

(52) U.S. Cl. CPC A61M 37/0015; A61M 5/3295; A61M 5/46; A61M 2037/0023; A61M 2037/0061; A61M 2205/3327

(57) ABSTRACT

A device comprising: a sleeve body defining an internal bore and terminating at a distal opening; a hollow microneedle defining an internal lumen and terminating at a distal pointed tip, wherein the microneedle is configured to be reciprocally displaceable within the internal bore such that the pointed tip extends and retracts through the distal opening; and a plunger having a shaft which is operable for advancing a seal along the internal lumen to deliver a quantity of a specified composition within the lumen along the internal lumen and through an outlet at a distal portion of the microneedle.

### CROSS-REFERENCE TO RELATED APPLICATIONS

This application claims the benefit of priority from U.S. Provisional Patent Application No. 63/446,390, filed Feb. 17, 2023, entitled, “MICRONEEDLE FOR INTRADERMAL FILLING,” the contents of which are hereby incorporated herein by reference in their entirety.

### FIELD OF THE INVENTION

The invention relates generally to devices and methods for delivery of dermal filler compositions into the skin of a subject using a microneedle device.

### BACKGROUND OF THE INVENTION

Skin is composed of the top layers of epidermis and dermis. Below these layers lies the hypodermis, also commonly referred to as subcutaneous fat layer, sub-cutis or subcutaneous tissue, which is not usually classified as a layer of skin.

The topmost layer of epidermis is made up of stratified squamous epithelium with an underlying basement membrane. It contains no blood vessels, and is nourished by diffusion from the dermis. This layer of skin functions as a barrier between the body and the external environment, keeping water in the body and preventing penetration of harmful chemicals and pathogens. The thickness of the epidermis in adult facial and neck skin is usually between 30 μm and 60 μm (micron, micrometer), depending on the specific location in the body. The thinnest epidermis is usually found in the posterior auricular, with a thickness of about 29.5 μm, while the thickest epidermis is usually found in the upper lip, with a thickness of about 62.6 μm. The average skin thickness in the face and neck (in areas were wrinkles, lines and folds are common) is 1.26 millimeter (mm).

The dermis lies below the epidermis and contains a number of structures including blood vessels, nerves, hair follicles, smooth muscle, glands and lymphatic tissue. The dermis (or corium) in facial and neck skin is typically 700-2000 μm thick, and is the major component of human skin. It is composed of a network of dense connective tissue, predominantly collagen fibrils providing support and elastin fibers providing flexibility.

The hypodermis lies below the dermis and is important for attaching the skin to the underlying bone and muscle as well as supplying it with blood vessels and nerves. The hypodermis is made up of loose connective tissue and elastin and contains fibroblasts, macrophages and adipocytes. The adipocytes play a major role in the fat storage function of the hypodermis. The fat serves as a filling material and as insulation of the body from the external environment.

Facial aging occurs as the result of several factors, among them are inherent changes within the skin, effects of gravity, activity of facial muscles leading to the formation of dynamic lines, skin loss or shift, bone loss, loss of tissue elasticity and exposure to harsh environmental conditions, particularly the sun or ultraviolet radiation and pollutants. The skin ages when the epidermis begins to thin, causing the junction with the dermis to flatten. Collagen decreases as a person ages and the bundles of collagen, which gives the skin turgor, become looser and lose strength. When the skin loses elasticity, it is less able to resist stretching. Coupled with gravity, muscle pull, and tissue changes, the skin begins to wrinkle. Water loss and breakdown of bonds between cells also reduces the barrier function of the skin, which can cause the skin's pore size to increase.

There have been efforts to develop and use compositions to correct defects in skin, such as scars and wrinkles, or to augment the tissue of a subject in order to improve the appearance of the skin, particularly facial skin. Currently, there are dozens of known dermal filling agents for skin augmentation which include autologous implantable materials, allogeneic products, xenogeneic products and synthetically derived products.

Available dermal fillers comprise biodegradable natural substances (such as collagen, gelatine, hyaluronic acid, dextran and dried acellular particulate dermal matrix), biodegradable synthetic polymers (such as poly-L-lactic acid, polyethylene oxide and carboxymethylcellulose), non-biodegradable synthetic polymers (such as polymethyl methacrylate, polyacrylamide, polyalkylimide and silicones) and combinations thereof.

Biocompatible ceramic skin augmentation materials, such as hydroxyapatite (Ca₅(PO₄)₃(OH)), are known to be efficient skin augmentation materials. Hydroxyapatite is a naturally occurring mineral form of calcium phosphate. Hydroxyapatite comprises the mineral constituent of bone, therefore rendering it biocompatible and non-immunogenic when introduced into the body of a subject. Of note, hydroxyapatite is biodegradable following the same metabolic pathways as bone debris resulting from common bone fractures, yet is semi-permanent, as it lasts up to 3 years when implanted into a subject. Moreover, when injected as small microspheres, hydroxyapatite acts as a scaffold that promotes new tissue formation similar to its surrounding environment. Inside skins such as the dermis, deposited particles of Hydroxyapatite support fibroblastic ingrowth and new collagen formation.

Skin augmentation products are typically injected with a needle just below the surface of the skin, at the site of the wrinkle, line, or fold (or scar or subcutaneous tissue to be enhanced). The products essentially plump up the skin from beneath the upper layers of skin. However, application of dermal fillers by injection or implantation is uncomfortable and possibly painful to the subject, and, furthermore, requires highly trained medical professionals to perform the procedure.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

There is provided, in an embodiment, a device comprising: a sleeve body defining an internal bore and terminating at a distal opening; a hollow microneedle defining an internal lumen and terminating at a distal pointed tip, wherein the microneedle is configured to be reciprocally displaceable within the internal bore such that the pointed tip extends and retracts through the distal opening; and a plunger having a shaft which is operable for advancing a seal along the internal lumen to deliver a quantity of a specified composition within the lumen along the internal lumen and through an outlet at a distal portion of the microneedle.

In some embodiments, the device further comprises a reservoir attached at a proximal end of the body sleeve and configured to store a predetermined amount of the specified composition.

In some embodiments, the microneedle comprises an aperture extending along a sidewall of the microneedle, wherein the aperture provides a communication between the reservoir and the internal lumen which allows for ingress of the quantity of the specified composition from the reservoir into the internal lumen.

In some embodiments, the pointed tip is configured to puncture a skin of a subject at an injection site.

In some embodiments, the distal opening is configured to rest against an external surface of the skin of the subject at the injection site, wherein the microneedle is configured to extend though the distal opening a predetermined distance into a dermal layer or hypodermal layer of the subject, to deliver at least a portion of the quantity of the specified composition at a desired depth location within the dermal or hypodermal layers.

In some embodiments, the microneedle is made of a material selected from a group consisting of: metal, plastic, a ceramic material, a silicone material, and any combination thereof.

In some embodiments, the microneedle has a cross-sectional shape selected from the group consisting of: circular, rectangular, oval, triangular, and polygonal.

In some embodiments, the pointed tip comprises a tip having an angle equal to between tip having an angle of convergence of between 10-60 degrees.

In some embodiments, the specified composition is a skin augmentation composition.

In some embodiments, the specified composition has a consistency that is liquid, viscous, semisolid, or solid.

In some embodiments, the specified composition comprises hydroxyapatite or hyaluronic acid.

In some embodiments, the specified composition comprises solid particles or solid spheres.

In some embodiments, the device further comprises one or more sensors configured to perform imaging or depth measurement of a local skin structure at the injection site, wherein the one or more sensors are selected from the group consisting of: an ultrasound sensor, an optical transducer, and a pressure gage force transducer.

In some embodiments, the device further comprises one or more imaging devices configured to perform imaging of a path of advancement of the device at the injection site.

In some embodiments, the outlet is located at the pointed tip.

There is also provided, in an embodiment, a method comprising: providing a device comprising a sleeve body defining an internal bore and terminating at a distal opening, a hollow microneedle defining an internal lumen and terminating at a distal pointed tip, wherein the microneedle is configured to be reciprocally displaceable within the internal bore such that the pointed tip extends and retracts through the distal opening, and a plunger having a shaft which is operable for advancing a seal along the internal lumen to deliver a quantity of a specified composition within the lumen along the internal lumen and through an outlet at a distal portion of the microneedle; locating the device at an injection site about a skin of a subject; displacing the microneedle within the internal bore such that the pointed tip extends a predetermined distance through the distal opening into a dermal layer or hypodermal layer of the skin of the subject; and operating the shaft to advance the seal along the internal lumen to deliver at least a portion of the quantity of the specified composition along the internal lumen and through the outlet at the pointed tip, into the dermal or hypodermal layers.

In some embodiments, the device further comprises a reservoir attached at a proximal end of the body sleeve and configured to store a predetermined amount of the specified composition.

In some embodiments, the microneedle comprises an aperture extending along a sidewall of the microneedle, wherein the aperture provides a communication between the reservoir and the internal lumen which allows for ingress of the quantity of the specified composition from the reservoir into the internal lumen.

In some embodiments, the pointed tip is configured to puncture the skin of the subject at the injection site.

In some embodiments, the distal opening is configured to rest against an external surface of the skin of the subject at the injection site, wherein the predetermined distance defines a desired depth location within the dermal or hypodermal layers for delivering the at least a portion of the quantity of the specified composition.

In some embodiments, the microneedle is made of a material selected from a group consisting of: metal, plastic, a ceramic material, a silicone material, and any combination thereof.

In some embodiments, the microneedle has a cross-sectional shape selected from the group consisting of: circular, rectangular, oval, triangular, and polygonal.

In some embodiments, the pointed tip comprises a tip having an angle equal to between tip having an angle of convergence of between 10-60 degrees.

In some embodiments, the specified composition is a skin augmentation composition.

In some embodiments, the specified composition has a consistency that is liquid, viscous, semisolid, or solid.

In some embodiments, the specified composition comprises hydroxyapatite or hyaluronic acid.

In some embodiments, the specified composition comprises solid particles or solid spheres.

In some embodiments, the device further comprises one or more sensors configured to perform imaging or depth measurement of a local skin structure at the injection site, wherein the one or more sensors are selected from the group consisting of: an ultrasound sensor, an optical transducer, and a pressure gage force transducer.

In some embodiments, the device further comprises one or more imaging devices configured to perform imaging of a path of advancement of the device at the injection site.

In some embodiments, the outlet is located at the pointed tip.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIGS. 1A-1F illustrate an exemplary microneedle device for intradermal delivery of dermal filler compositions into the dermal tissue of a subject, according to some embodiments of the present disclosure;

FIGS. 2A-2H illustrate the operational steps of a method for intradermal delivery of dermal filler compositions into the dermal tissue of a subject using a microneedle device of the present disclosure, according to some embodiments of the present disclosure;

FIG. 3 is a flowchart of the operational steps of a method for intradermal delivery of dermal filler compositions into the dermal tissue of a subject using a microneedle device of the present disclosure, according to some embodiments of the present disclosure;

FIGS. 4A-4C illustrate additional exemplary embodiments of a microneedle device for intradermal delivery of dermal filler compositions into the dermal tissue of a subject, according to some embodiments of the present disclosure; and

FIG. 5 is a schematic illustration of an exemplary microneedle system for intradermal delivery of dermal filler compositions into the dermal tissue of a subject using a microneedle device of the present disclosure, according to some embodiments of the present disclosure.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. As used herein, the term “dermal” refers to the dermis layer of the skin.

Disclosed herein are a microneedle device and a method for using the microneedle device of the present disclosure for intradermal delivery of dermal filler compositions into the dermal tissue of a subject. In accordance with the method of the invention, a microneedle device of the present disclosure is deployed along a predefined line or wrinkle, where dermal filling, cosmetic repair or augmentation of tissue is desired, and a quantity of dermal filler composition is injected through the present microneedle device to one or more predetermined intradermal depth locations.

In some embodiments, the present microneedle device provides for exact intradermal filling of undesired lines, wrinkles, depressed scars and folds of a subject's skin, using one of several suitable biocompatible filling materials that may be injected into the dermis layer or hypodermis layer of a subject's facial or neck skin In some embodiments, the present microneedle device is particularly suited for accurate and precise delivery of viscous, dense, semisolid, or solid augmentation materials (such as calcium hydroxyapatite spheres in powder form).

In some embodiments, the present microneedle device provides for precise and accurate delivery of an augmentation composition into predetermined deficient areas of the dermis and hypodermis target tissues, thereby preventing waste of skin augmentation material, avoiding the need for repeated or multiple administration cycles, and reducing any undesirable side-effects associated with delivery into the epidermis, such as the appearance of small solid lumps on the surface of the treated skin, which cause the skin to appear bumpy and uneven, and when injection is superficial, it may ulcerate the epidermis and become a source of infection and inflammation.

In some embodiments, the present microneedle device provides for exact localization of injection depth, based on pre-measuring skin depth along a predefined line or wrinkle where dermal filling is desirable. In some embodiments, such pre-measuring may be achieved using an ultrasound sensor, e.g., a high-resolution ultrasound (e.g., having a frequency that is greater than 10 MHz), to obtain high-resolution imaging of local skin structures at a desired injection site. In some embodiments, such pre-measuring allows the medical practitioner performing the procedure to accurately measure the depth of the dermis layer at the injection site, and to determine one or more optimum injection depths at the measured injection site.

The present invention provides, in one aspect, a microneedle device comprising a microneedle for administering a skin augmentation composition intradermally, e.g., to the dermis layer and/or hypodermis layer of human facial or neck skin.

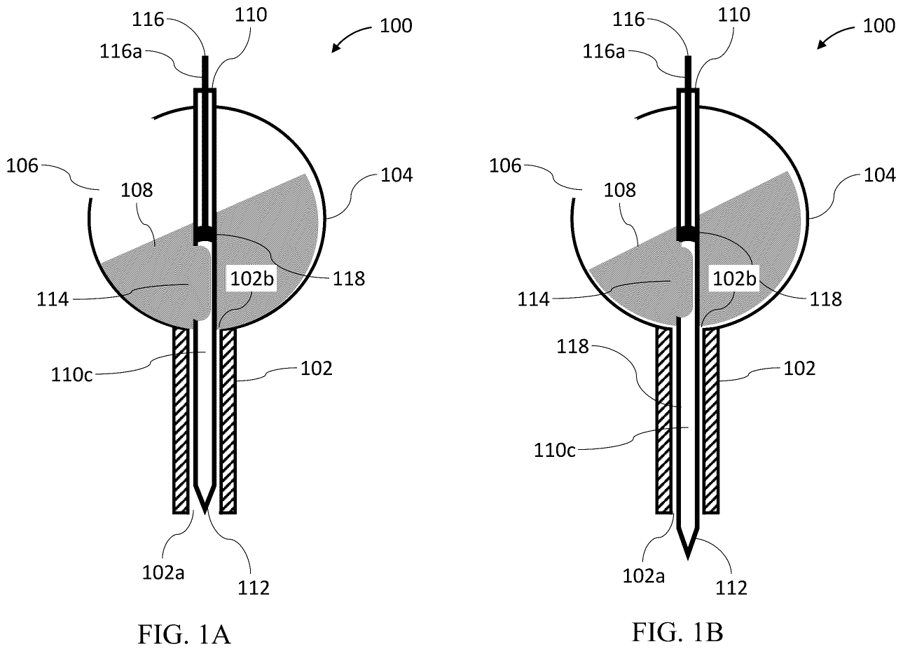

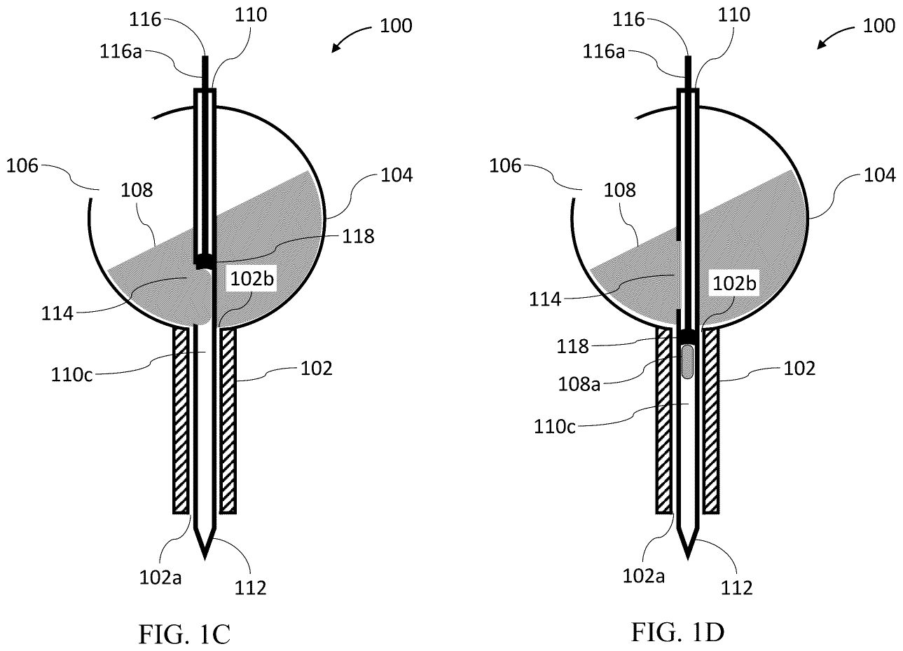

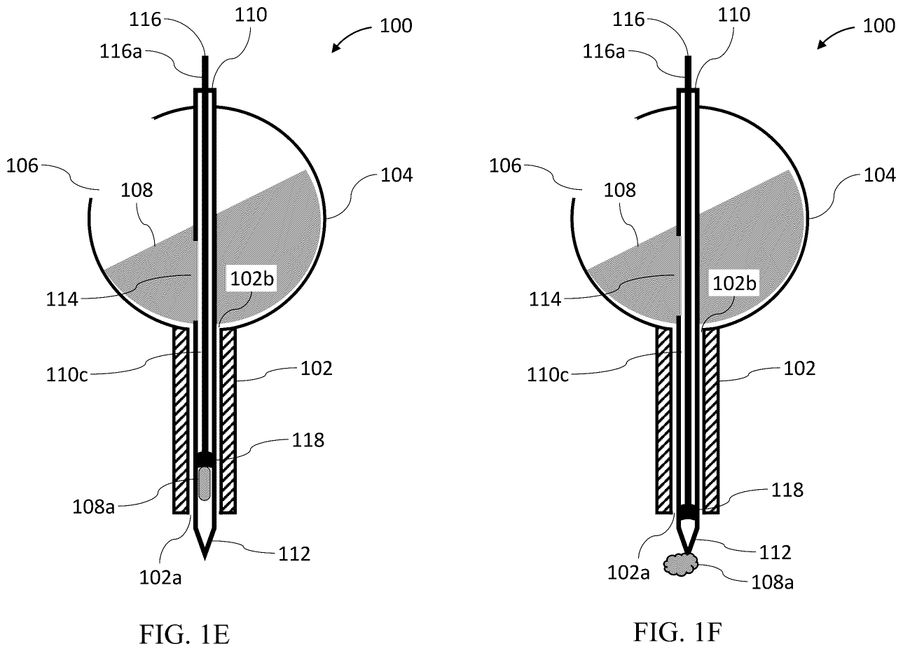

Reference is made to FIGS. 1A-1F which illustrate an exemplary microneedle device 100 for intradermal delivery of dermal filler compositions into the dermal tissue of a subject, according to some embodiments of the present disclosure.

In some embodiments, microneedle device 100 of the present disclosure comprises at least:
A syringe sleeve body 102 defining an internal bore and terminating at a distal opening 102a.
A reservoir 104 attached at a proximal end 102b of sleeve body 102 and configured to store a predetermined amount of skin augmentation composition 108 therein, wherein reservoir 104 has a refill opening 106.
A hollow microneedle 110 extending within reservoir 104 and through proximal end 102b into the internal bore defined by sleeve body 102, wherein microneedle 110 is configured to be reciprocally displaceable along the internal bore of sleeve body 102, such that a pointed distal tip 112 of microneedle 100 may extend and retract through distal opening 102a of sleeve body 110.
A port or aperture 114 extending along a side wall of microneedle 110 and located so as to provide communication between reservoir 104 and a lumen 110c of microneedle 110, such that skin augmentation composition 108 may flow or ingress into lumen 110c through port 114.
A plunger 116 having a shaft 116a for advancing a seal 118 along the lumen 110c of microneedle 110, so as to (i) collect a quantity 108a of skin augmentation composition 108 communicated into lumen 110c of microneedle 110 through port 114, and (ii) deliver the quantity 108a of skin augmentation composition 108 along the lumen 110c of microneedle 110 and through an outlet at pointed tip 112. Plunger shaft 116a is formed with a plunger extension extending in a sealable engagement proximally from a proximal end of microneedle 110.

As used herein, the term “microneedle” refers to protruding structures designed to pierce the skin and facilitate delivery of various types of compounds into tissue. According to some embodiments, microneedles facilitate delivery of the composition of the invention to dermal and/or hypodermal compartments of the skin.

The principles and operation of microneedle device 100, according to some embodiments of the present disclosure, will now be discussed with continued reference to FIGS. 1A-1F.

In FIG. 1A, plunger 116 and seal 118 are shown in an initial position, wherein seal 118 is positioned proximally of port 114. Similarly, pointed tip 112 of microneedle 110 is in a retracted position within sleeve body 110, such that pointed tip 112 does not extend beyond distal opening 102a.

Microneedle device may be deployed along a predefined line or wrinkle of a subject's skin, where dermal filling, cosmetic repair or augmentation of tissue is desired. Once positioned at a desired injection site, microneedle 110 may be advanced distally, as shown in FIG. 1B, such that pointed tip 112 extends a predetermined distance from distal end 102a of sleeve body 102. Thus, with distal opening 102a of sleeve 102 resting against an external surface of the skin at the injection site, pointed tip 112 penetrates the skin to reach the predetermined injection depth, determined by the distance by which pointed tip 112 is extended through opening 102a. In some embodiments, the advancing distance of microneedle 110 through opening 102a may be predetermined based on pre-measuring of the skin structure at the injection site, to accurately measure the depth of the dermis layer at the injection site and to determine one or more optimum injection depths at the measured injection site.

In some embodiments, as shown in FIGS. 1C-1E, plunger 116 may then be advanced to drive seal 118 in the distal direction, so as to collect a quantity 108a of skin augmentation composition 108 which flows or ingresses into lumen 110c of microneedle 110 from reservoir 104 through port 114. In some embodiments, plunger 116 drives seal 118 to deliver quantity 108a distally through lumen 110c of microneedle 110, towards pointed tip 112. In FIG. 1F, seal 118 delivers quantity 108a through an outlet of pointed tip 112 at the predetermined injection depth.

In certain embodiments, microneedle 110 is 500, 1000, 1500, 2000, 2500, 3000, 4000, 5000, 6000 or 7000 μm in height. Each possibility represents a separate embodiment of the invention.

In certain embodiments, microneedle 110, sleeve body 102, and/or plunger 116 are each formed of a rigid material that is selected from a group consisting of metal, a metal alloy, a plastic, a ceramic material, a silicone material and a combination thereof. In certain embodiments, the metal is stainless steel. Each possibility represents a separate embodiment of the invention.

In certain embodiments, microneedle 110 has a cross-sectional shape selected from the group consisting of a circular, rectangular, oval, triangular, and polygonal. Each possibility represents a separate embodiment of the invention.

In certain embodiments, pointed tip 112 has the same diameter as the diameter of microneedle 110. In certain embodiments, pointed tip 112 section has a larger diameter than the total diameter of microneedle 110. In certain embodiments, pointed tip 112 has a diameter that is about 5%, about 10%, about 15% or about 20% larger than the diameter of microneedle 110. In certain embodiments, pointed tip 112 defines a tapered or conical tip portion having an angle of convergence of between 10-60 degrees, e.g., a 10 degree angle, a 20 degree angle, a 30 degree angle, a 40 degree angle, a 50 degree angle, or a 60 degree angle. Each possibility represents a separate embodiment of the invention. In certain embodiments, pointed tip 112 has a shape selected from the group consisting of: a cone, a taper, a pyramid, a triangular pyramid and a polygonal pyramid. Each possibility represents a separate embodiment of the invention.

In certain embodiments, pointed tip 112 is hollow and terminates in an outlet or opening, to allow delivery of augmentation composition 108 therethrough. In certain embodiments, pointed tip 112 is blocked or sealed and does not terminate in an outlet, to allow delivery of augmentation composition through holes or slits in microneedle 110 itself.

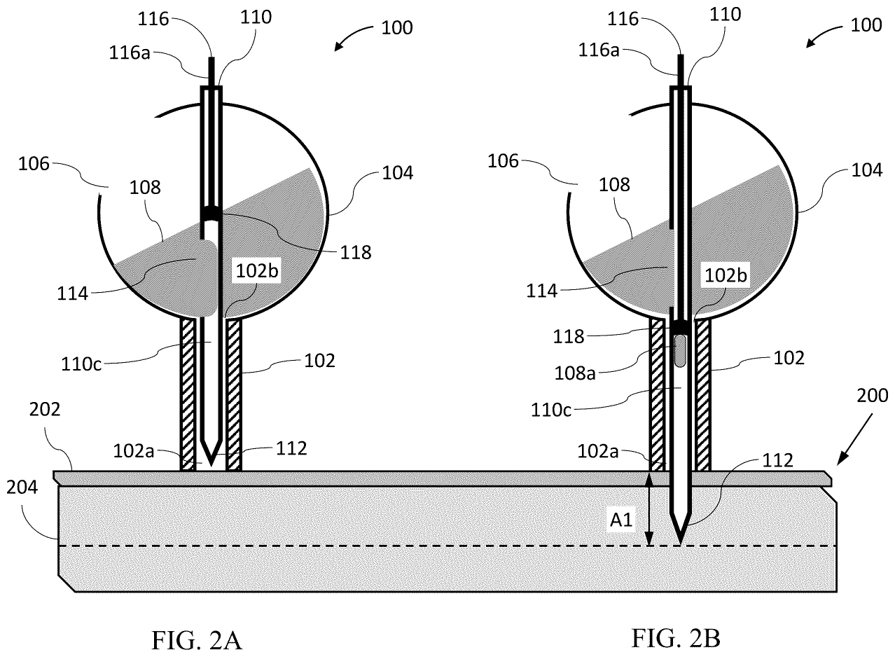

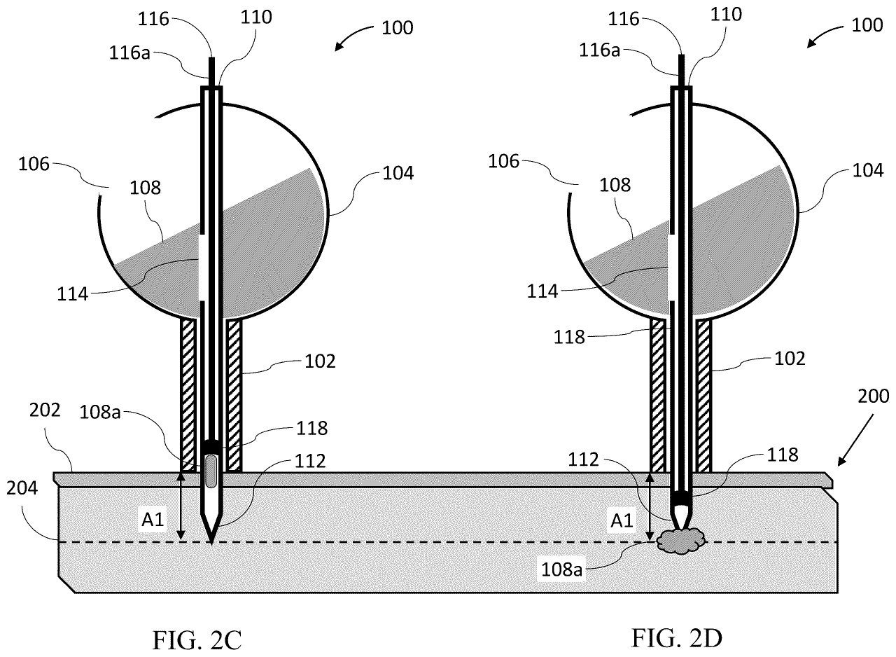

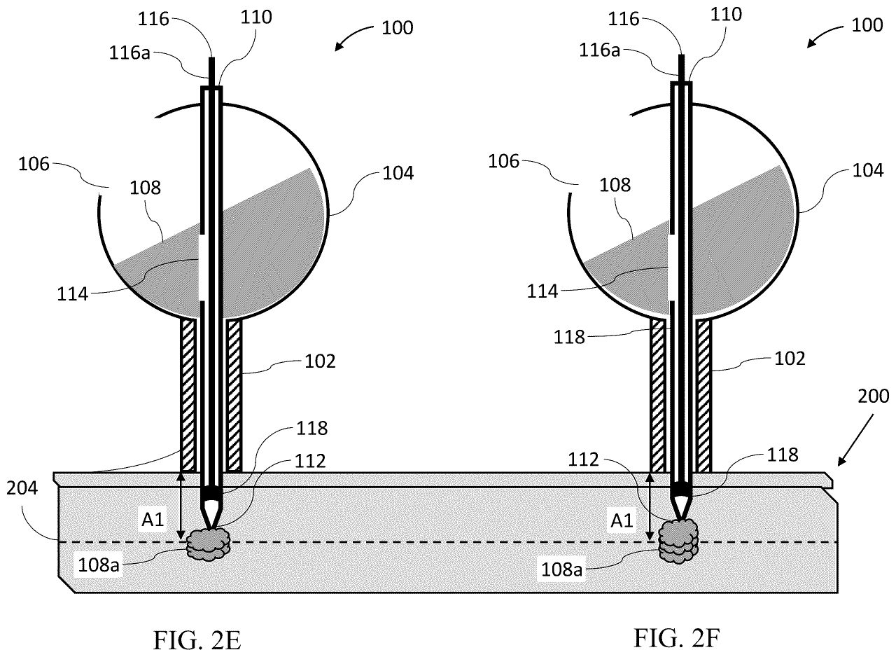

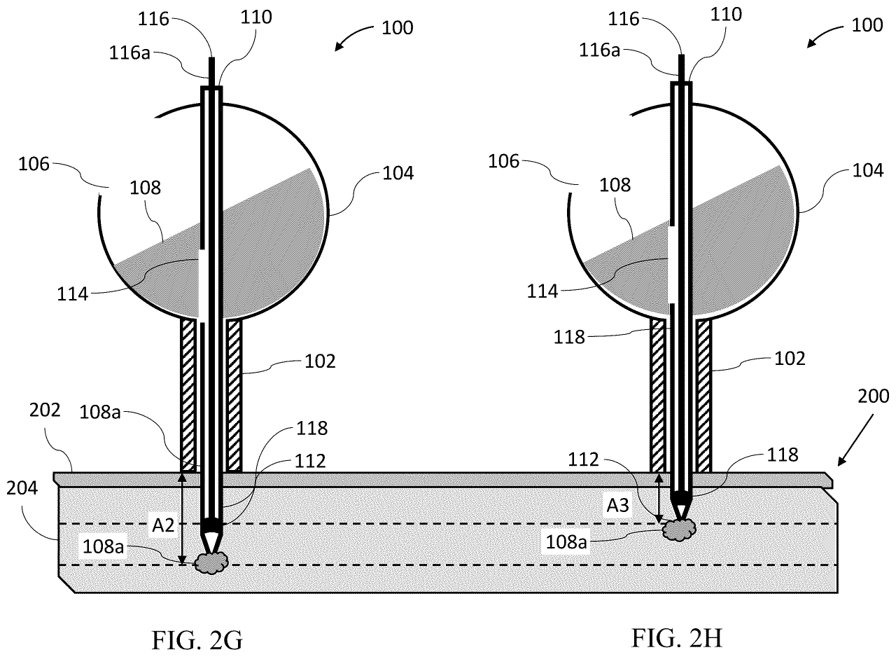

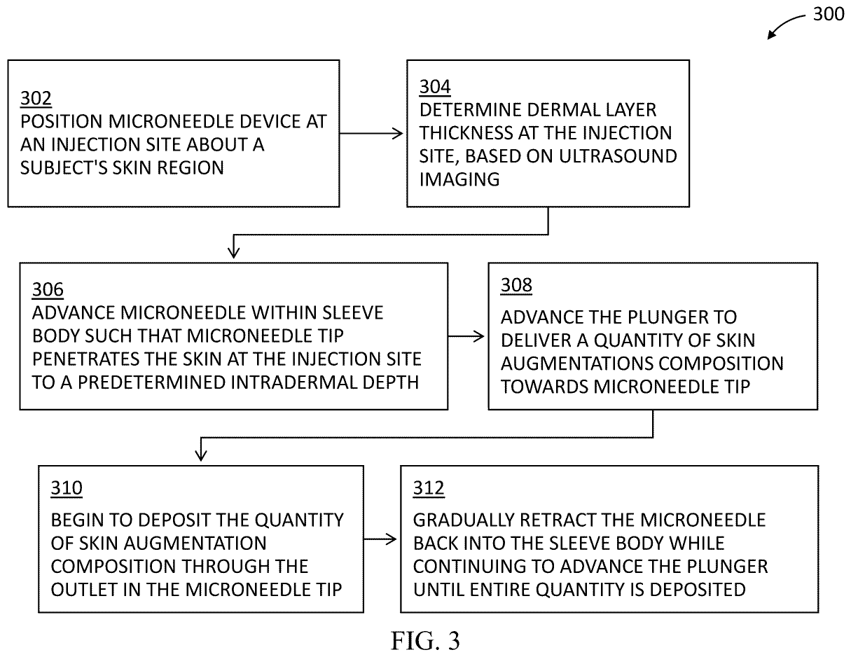

The present invention further provides, in another aspect, a method 300 for intradermal delivery of dermal filler compositions into the dermal tissue of a subject using a microneedle device (such as microneedle devices variation 100 or variations 120, 130, and/or 140). The various steps of method 300 will be discussed with reference to the flowchart of FIG. 3 and to FIGS. 2A-2D. Method 300 may either be performed in the order they are presented or in a different order (or even in parallel), as long as the order allows for necessary conditions or results to a certain step to be obtained from an earlier step.

Although FIGS. 2A-2D illustrate the steps of method 300 with reference to exemplary microneedle device 100, the particulars of method 300 are equally applicable in the case of exemplary microneedle device variations 120, 130, and/or 140.

Method 300 begins in step 302 (FIG. 2A), wherein microneedle device 100 may be deployed along a predefined line or wrinkle about a subject's skin region 200, comprising epidermis layer 202 and dermis/deeper level hypodermis layer 204. In some embodiments, microneedle device 100 may be in an initial position, wherein seal 118 is positioned proximally of port 114. Similarly, pointed tip 112 of microneedle 110 is in a retracted position within sleeve body 110, such that pointed tip 112 does not extend beyond distal opening 102a.

In some embodiments, in step 304, ultrasound imaging of the skin region 200 structure at the injection site may be performed, to accurately measure the depth of the dermis/hypodermis layer 204 at the injection site and to predetermine one or more optimum injection depths at the measured injection site (generally, at or about a midpoint in the depth dimension of the dermis/hypodermis layer).

In step 306 (FIG. 2B), microneedle 110 may be advanced distally, as shown in FIG. 1B, such that pointed tip 112 extends the predetermined distance A1 (as determined in step 304) from distal end 102a of sleeve body 102. Thus, with distal opening 102a of sleeve 102 resting against an external surface of the skin at the injection site, pointed tip 112 penetrates the skin to reach the predetermined intradermal injection depth (marked by a horizontal dashed line), determined by the distance Al by which pointed tip 112 is extended through opening 102a.

In step 308 (FIG. 2C), plunger 116 may then be advanced to drive seal 118 in the distal direction, so as to collect a quantity 108a of skin augmentation composition 108 which flows or ingresses into lumen 110c of microneedle 110 from reservoir 104 through port 114. In some embodiments, plunger 116 drives seal 118 to deliver quantity 108a distally through lumen 110c of microneedle 110, towards pointed tip 112.

In step 310 (FIG. 2D), plunger 116 continues to be advanced towards pointed tip 112, until seal 118 delivers quantity 108a through an outlet of pointed tip 112 at or about the predetermined injection depth A1, and quantity 108a begins to be deposited at or about the predetermined injection depth A1, e.g., at least a first portion of quantity 108a has been deposited at or about depth A1.

In step 312 (FIGS. 2E-2F), microneedle 110 may begin to be gradually retracted back through distal opening 102a of sleeve body 102, such that pointed tip 112 may be retracted back along a path of retraction which extends about depth portion of dermis layer 204 at the injection site. As microneedle 110 is gradually retracted back, steps 308-310 may be repeated one or more times, to deposit additional one or more portions of quantity 108a along the path of retraction, until the full amount of quantity 108a has been deposited about the predetermined depth in its entirety.

It is noted that plunger 116 may be operated independently of microneedle 110, wherein plunger 116 and microneedle 110 are capable of relative motion relative to one another, and relative to sleeve body 102, such that, for example, microneedle 110 may be gradually retracted while plunger 116 may be reciprocally movable (i.e., advanced and retracted) within the bore of and relative to microneedle 110 and vice versa.

For example, in some embodiments, optionally, during step 312, plunger 116 may be retracted one or more times back to its initial position wherein seal 118 is located proximally to port 114, to optionally collect additional quantities of the skin augmentations composition to supplement quantity 108a, as may be deemed necessary or desirable by the medical practitioner performing the procedure. Plunger 116 may then be advanced as described in steps 308-310 above, to deliver the additional supplemental quantities at of about the predetermined depth.

In some embodiments, one or more of plunger 116 and microneedle 110 may be operated and movable via a variety of hand-operated and/or automated control means and manipulators of different types using a wide variety of mechanisms and electromechanical elements including, for example, angular and linear motors, optical or electromagnetic position encoders, gimbals, linkages, pulleys, cables, drive belts and bands, and gears.

With reference to FIGS. 2G-2H, steps 306-312 of method 300 may be repeated two or more times at the injection site, to the deliver skin augmentations composition at two or more desirable depths within dermis-hypodermis layer 204, e.g., depth A2 (FIG. 2G) and/or depth A3 (FIG. 2H).

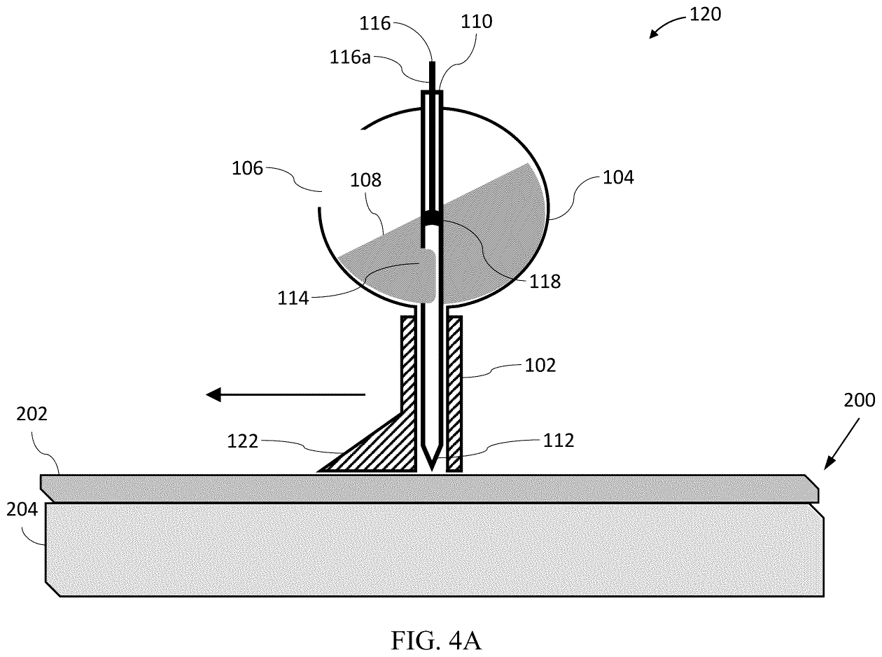

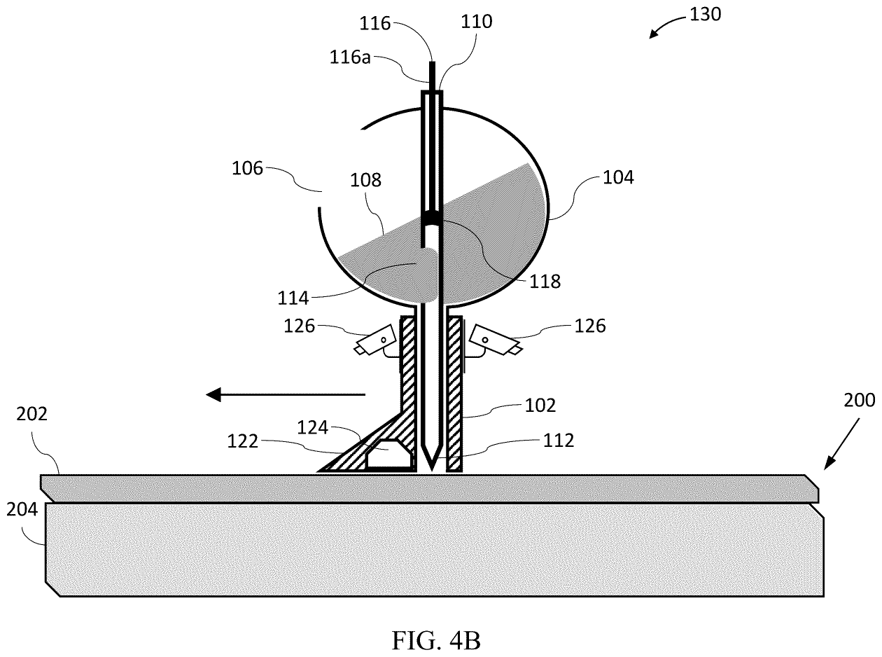

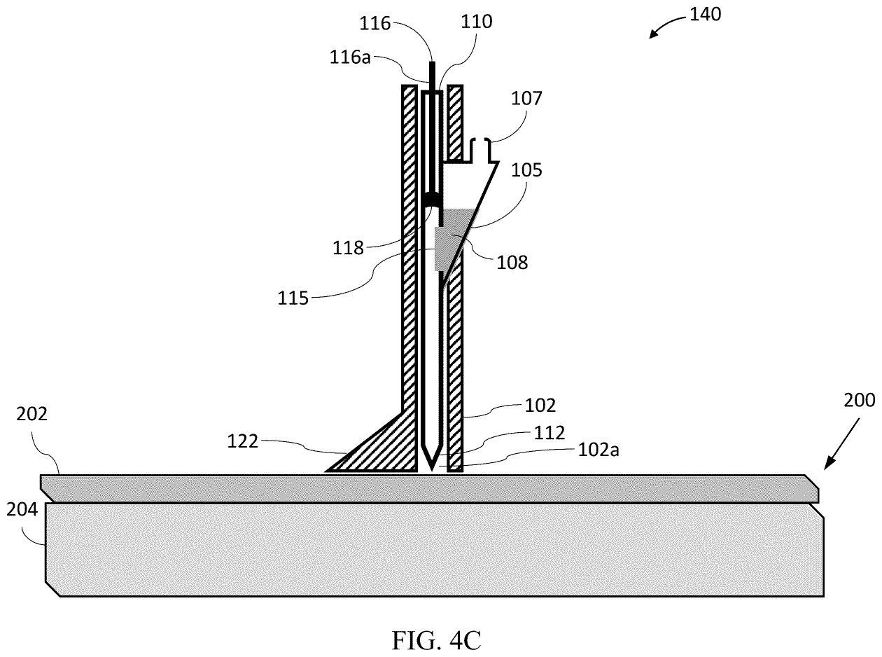

Reference is made to FIG. 4A which illustrates an exemplary variation 120 of microneedle device 100 for intradermal delivery of dermal filler compositions into the dermal tissue of a subject, according to some embodiments of the present disclosure. Microneedle device variation 120 may be substantially similar to microneedle device 100 described hereinabove, and comprises a sleeve body 102; a reservoir 104 with a refill opening 106 for storing skin augmentations composition 108; a microneedle 110 with a pointed tip 112 and a port 114; and a plunger 116 with a shaft 116a and a seal 118. The principles and operation of microneedle device variation 120 are substantially similar to those of microneedle device 100, as described above with reference to FIGS. 1A-1F.

In some embodiments, microneedle device variation 120 comprises a nose projection 122 extending laterally from a distal end of sleeve body 102. Nose projection 122 is configured to provide a visual indication as to the precise position of pointed tip 112 when microneedle device variation 120 is deployed along a predefined line or wrinkle about skin region 200, where dermal filling, cosmetic repair or augmentation of tissue is desired. In some embodiments, nose projection 122 may further provide for smoothing of skin region 200 ahead of the microneedle device variation 120, as the microneedle device variation 120 is moved along the predefined path about skin region 200, e.g., in the direction marked by an arrow. In some embodiments, nose projection 122 may also serve as a pointer or an arrow to assist the medical practitioner performing the procedure in navigating the device precisely along the wrinkle or line to be treated.

Reference is made to FIG. 4B which illustrates an exemplary variation 130 of microneedle device 110 for intradermal delivery of dermal filler compositions into the dermal tissue of a subject, according to some embodiments of the present disclosure. Microneedle device variation 130 may be substantially similar to microneedle device 100 described hereinabove, and comprises a sleeve body 102; a reservoir 104 with a refill opening 106 for storing skin augmentations composition 108; a microneedle 110 with a pointed tip 112 and a port 114; a plunger 116 with a shaft 116a and a seal 118; and a nose projection 122. The principles and operation of microneedle device variation 130 are substantially similar to those of microneedle device 100, as described above with reference to FIGS. 1A-1F.

In some embodiments, microneedle device variation 130 comprises one or more imaging devices (e.g., video cameras) 126 configured to image the skin region 200. In some embodiments, microneedle device variation 130 comprises a nose projection 122 extending laterally from a distal end of sleeve body 102. Nose projection 122 is configured to house one or more ultrasound sensors or other sensors 124.

In some embodiments, ultrasound sensor(s) 124 may be high-resolution ultrasound sensor(s) (e.g., having a frequency that is greater than 10 MHz), to obtain high-resolution imaging of local skin region 200 structures at the desired injection site. In some embodiments, such pre-measuring allows the medical practitioner performing the procedure to accurately measure the depth of the dermis layer at the injection site, and to determine one or more optimum injection depths at the measured injection site.

In some embodiments, imaging device(s) 126, optionally having magnification capability, may be configured to assist in pinpointing the exact location of microneedle device variation 130 when deployed along a predefined path about subject's skin region 200, e.g., in the direction marked by an arrow.

Reference is made to FIG. 4C which illustrates a variation 140 of microneedle device 100 for intradermal delivery of dermal filler compositions into the dermal tissue of a subject, according to some embodiments of the present disclosure. Microneedle device variation 140 comprises a sleeve body 102; a tapering or conical reservoir 105 with a refill opening 107 for storing skin augmentations composition 108; a microneedle 110 with a pointed tip 112 and a port 115; a plunger 116 with a shaft 116a and a seal 118; and a nose projection 122. The principles and operation of microneedle device variation 140 are substantially similar to those of microneedle device 100, as described above with reference to FIGS. 1A-1F.

In some embodiments, variation 140 of microneedle device 100 comprises at least:
A syringe sleeve body 102 defining an internal bore and terminating at a distal opening 102a.
A tapering or conical reservoir 105 located along a side wall of sleeve body 102 and configured to store a predetermined amount of skin augmentation composition 108 therein, wherein reservoir 105 has a refill opening 107.
A port or aperture 115 extending along a side wall of microneedle 110 and located so as to provide communication between reservoir 105 and a lumen 110c of microneedle 110, such that skin augmentation composition 108 may flow or ingress into lumen 110c into port 115.
A hollow microneedle 110 extending within the internal bore defined by sleeve body 102, wherein microneedle 110 is configured to be reciprocally displaceable along the internal bore of sleeve body 102, such that a pointed distal tip 112 of microneedle 100 may extend and retract through distal opening 102a of sleeve body 110.
A plunger 116 having a shaft 116a for advancing a seal 118 along the lumen 110c of microneedle 110, so as to (i) collect a quantity 108a of skin augmentation composition 108 communicated into lumen 110c of microneedle 110 through port 115, and (ii) deliver the quantity of skin augmentation composition along the lumen 110c of microneedle 110 and through an outlet at pointed tip 112. Plunger shaft 116a is formed with a plunger extension extending in a sealable engagement proximally from a proximal end of microneedle 110.

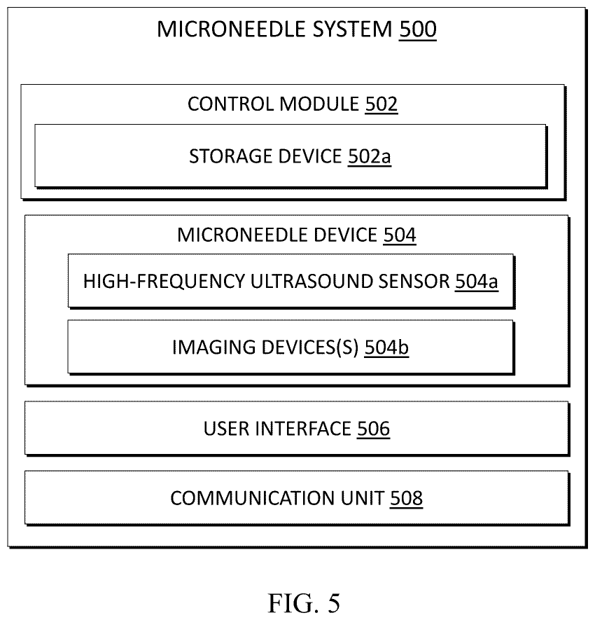

FIG. 5 is a schematic illustration of an exemplary microneedle system 500 for intradermal delivery of dermal filler compositions into the dermal tissue of a subject using a microneedle device of the present disclosure. In some embodiments, microneedle system 500 is configured for intradermal delivery of dermal filler compositions into the dermal tissue of a subject, according to some embodiments of the present disclosure. In some embodiments, microneedle system 500 comprises a microneedle device 504, which may be embodies in any one of exemplary microneedle device 100 shown in FIGS. 1A-1F and 2A-2H, and/or exemplary microneedle device variation 120 in FIGS. 4A, 130 in FIG. 4B, and/or 140 in FIG. 4C). Typically, the microneedle device 504 comprises one or more high frequency ultrasound sensor(s) 504a and one or more imaging device(s) 504b.

In some embodiments, microneedle system 500 includes a control module 502 configured to control the operation of microneedle system 500 and microneedle device 504. Control module 502 may include one or more hardware processor(s), a random-access memory (RAM), and one or more non-transitory computer-readable storage device(s), such as storage device 502a. Components of control module 502 may be co-located or distributed, or control module 502 may be configured to run as one or more cloud computing ‘instances,’ ‘containers,’ ‘virtual machines,’ or other types of encapsulated software applications, as known in the art.

Storage device 502a may have stored thereon program instructions and/or components configured to operate control module 502. The program instructions may include one or more software modules, which may include an operating system having various software components and/or drivers for controlling and managing general system tasks (e.g., memory management, storage device control, power management, etc.), and facilitating communication between various hardware and software components. Control module 502 may operate by loading instructions of the various software modules into its RAM as they are being executed by the hardware processor(s) comprising control module 502.

Control module 502 as described herein is only an exemplary embodiment of the present invention, and in practice may be implemented in hardware only, software only, or a combination of both hardware and software. Control module 502 may have more or fewer components and modules than shown, may combine two or more of the components, or may have a different configuration or arrangement of the components. Control module 502 may include any additional component enabling it to function as an operable computer system, such as a motherboard, data busses, power supply, a network interface card, a display, an input device, etc. Moreover, components of control module 502 may be co-located or distributed (e.g., on an external control or computing device), or the system may be configured to run as one or more cloud computing ‘instances,’ ‘containers,’ ‘virtual machines,’or other types of encapsulated software applications, as known in the art.

In some embodiments, microneedle system 500 comprises a user interface 506 which may include a display screen, which is optionally a touchscreen. User interface 506 which is used to provide outputs to a user, and/or receive inputs from the user.

In some embodiments, microneedle system 500 or any module thereof is configured to communicate with one or more external computing devices (not shown), which may include a computer processor of a local or a remote computing device or a remote server, e.g., a cloud-based remote server. For example, an external computing devices may receive data from control module 502 and may then send data and/or instructions to control module 502.

In some embodiments, microneedle system 500 comprises a communication unit 508 configured to send information to, and/or receive information from, an external computing device. The sending and the receiving of information may be implemented wirelessly and/or by wire.

In certain embodiments, the skin augmentation composition is solid at room temperature. In certain embodiments, the skin augmentation composition is solid at a temperature of 10° C., 20° C., 30° C., 40° C., or 50° C. In certain embodiments, the skin augmentation composition is semi solid at room temperature, or at a temperature of 10° C., 20° C., 30° C., 40° C., or 50° C.

In some embodiments, the skin augmentation composition comprises a skin augmentations composition substance or material and a dispersant. In certain embodiments, the skin augmentation composition substantially consists of the biocompatible skin augmenting material and a biocompatible dispersant. In certain embodiments, the skin augmentation composition comprises no dispersant.

In some embodiments, the skin augmentation composition is solid at room temperature, the skin augmenting material is solid at room temperature and the dispersant is solid at room temperature. In certain embodiments, the skin augmentation composition is solid at room temperature, the skin augmenting material is semi-solid at room temperature and the dispersant is solid at room temperature. In certain embodiments, the skin augmentation composition is solid at room temperature, the skin augmenting material is solid at room temperature and the dispersant is semi-solid at room temperature. In certain embodiments, the skin augmentation composition is solid at room temperature, the skin augmenting material is semi-solid at room temperature and the dispersant is semi-solid at room temperature. In certain embodiments, the skin augmentation composition is semi-solid at room temperature, the skin augmenting material is solid at room temperature and the dispersant is solid at room temperature. In certain embodiments, the skin augmentation composition is semi-solid at room temperature, the skin augmenting material is semi-solid at room temperature and the dispersant is solid at room temperature. In certain embodiments, the skin augmentation composition is semi-solid at room temperature, the skin augmenting material is solid at room temperature and the dispersant is semi-solid at room temperature. In certain embodiments, the skin augmentation composition is semi-solid at room temperature, the skin augmenting material is semi-solid at room temperature and the dispersant is semi-solid at room temperature. In certain embodiments, the skin augmentation composition is liquid at room temperature In certain embodiments, the skin augmentation composition is solid at room temperature, comprises about 50% to about 75% by weight of the biocompatible skin augmenting material, and about 25% to about 50% by weight of the biocompatible dispersant, wherein at least about 20% of the total volume of the microneedle is filled with the skin augmentation composition.

In certain embodiments, the skin augmentation composition is solid at room temperature, comprises about 60% to about 65% by weight of the biocompatible skin augmenting material, and about 35% to about 40% by weight of the biocompatible dispersant, wherein about 40% to about 50% of the total volume of the microneedle is filled with the skin augmentation composition.

In certain embodiments, the skin augmentation composition comprises at least about 30% by weight of the biocompatible skin augmenting material. In certain embodiments, the skin augmentation composition comprises at least about 35% by weight of the biocompatible skin augmenting material. In certain embodiments, the skin augmentation composition comprises at least about 40% by weight of the biocompatible skin augmenting material. In certain embodiments, the skin augmentation composition comprises at least about 45% by weight of the biocompatible skin augmenting material. In certain embodiments, the skin augmentation composition comprises at least about 50% by weight of the biocompatible skin augmenting material. In certain embodiments, the skin augmentation composition comprises at least about 55% by weight of the biocompatible skin augmenting material. In certain embodiments, the skin augmentation composition comprises at least about 60% by weight of the biocompatible skin augmenting material.

In certain embodiments, the skin augmentation composition comprises at least about 20% by weight of the at least one biocompatible dispersant. In certain embodiments, the skin augmentation composition comprises at least about 25% by weight of the at least one biocompatible dispersant. In certain embodiments, the skin augmentation composition comprises at least about 30% by weight of the at least one biocompatible dispersant. In certain embodiments, the skin augmentation composition comprises at least about 30% by weight of the at least one biocompatible dispersant. In certain embodiments, the skin augmentation composition comprises at least about 35% by weight of the at least one biocompatible dispersant.

In certain embodiments, the skin augmentation composition comprises at least about 30%, at least about 35%, at least about 40%, at least about 45%, at least about 50%, at least about 55% or at least about 60% by weight of the biocompatible skin augmenting material; and at least about 20%, at least about 25%, at least about 30% or at least about 35% by weight of the at least one biocompatible dispersant. Each possibility represents a separate embodiment of the invention.

In certain embodiments, the skin augmentation composition comprises 4% by weight of the biocompatible skin augmenting material for every 3% by weight of the at least one biocompatible dispersant. In certain embodiments, the skin augmentation composition comprises 5% by weight of the biocompatible skin augmenting material for every 3% by weight of the at least one biocompatible dispersant. In certain embodiments, the skin augmentation composition comprises 6% by weight of the biocompatible skin augmenting material for every 3% by weight of the at least one biocompatible dispersant.

In certain embodiments, the skin augmentation composition comprises about 50% to about 75% by weight of the biocompatible skin augmenting material, and about 25% to about 50% by weight of the biocompatible dispersant. In certain embodiments, the skin augmentation composition comprises about 60% to about 65% by weight of the biocompatible skin augmenting material, and about 35% to about 40% by weight of the biocompatible dispersant. In certain embodiments, the skin augmentation composition comprises about 62.5% by weight of the biocompatible skin augmenting material, and about 37.5% by weight of the biocompatible dispersant.

In certain embodiments, at least about 20% of the total volume of the microneedle is filled with the skin augmentation composition. In certain embodiments, at least about 30% of the total volume of the microneedle is filled with the skin augmentation composition. In certain embodiments, at least about 40% of the total volume of the microneedle is filled with the skin augmentation composition. In certain embodiments, at least about 50% of the total volume of the microneedle is filled with the skin augmentation composition. In certain embodiments, about 40% to about 50% of the total volume of the microneedle is filled with the skin augmentation composition.

In certain embodiments, the biocompatible dispersant disperses at least a portion of the skin augmenting material into the dermis layer, into the hypodermis layer, or into both the dermis layer and the hypodermis layer. In certain embodiments, the biocompatible dispersant disperses at least a portion of the skin augmenting material into both the dermis layer and the hypodermis layer.

In certain embodiments, the biocompatible skin augmenting material is hydroxyapatite. In certain embodiments, the biocompatible skin augmenting material is hyaluronic acid. In certain embodiments, the biocompatible skin augmenting material is in the form of solid particles or solid spheres. In certain embodiments, about 10% of the particles or spheres are up to about 15 μm to about 35 μm in diameter. In certain embodiments, about 50% of the particles or spheres are up to about 35 μm to about 50 μm in diameter. In certain embodiments, about 90% of the particles or spheres are up to about 50 μm to about 70 μm in diameter. In certain embodiments, about 10% of the particles or spheres are up to about 26 μm in diameter. In certain embodiments, about 50% of the particles or spheres are up to about 41 μm in diameter. In certain embodiments, about 90% of the particles or spheres are up to 64 μm in diameter.

In certain embodiments, the biocompatible dispersant is a water-soluble polymer. In certain embodiments, the water-soluble polymer is polyethylene glycol (PEG), polyethylene oxide (PEO) or polyoxyethylene (POE). In certain embodiments, the water-soluble polymer has a molecular weight in the range of about 1000, about 2000, about 3000, about 4000, about 5000, about 6000, about 7000, about 8000, about 9000 or about 10000 to about 10000, about 11000, about 12000, about 13000, about 14000, about 15000, about 16000, about 17000, about 18000 or 19000 gram/mole. Each possibility represents a separate embodiment of the invention. In certain embodiments, the water-soluble polymer is PEG 12,000.

The present invention provides, for the first time, a microneedle-based applicator for delivery of a skin augmentation composition to the skin of a subject, the composition comprising at least one biocompatible filler material. The applicators of the invention provide an efficient, comfortable and easy-to-use delivery system for skin augmentation compositions. The present invention further provides delivery methods of skin augmentation compositions to the skin of a subject. The methods of the invention enable, inter alia, filling of undesired folds, wrinkles, or lines in a subject's skin. According to some embodiments, the methods of the invention enable a subject to use the applicators and methods of the invention without the help of a trained medical professional. According to other embodiments, the applicators of the invention may be supplied as disposable strips or patches.

Non-limiting examples of a skin defect or deficiency, according to some embodiments of the present invention, are selected from the group consisting of: undesired lines, wrinkles, folds, depressed scars, areas of skin or sub cutis deficiency or a combination thereof.

As used herein, the terms “composition”, “the composition of the invention” “augmentation composition”, “a soft tissue augmentation composition” and “skin augmentation composition” are used interchangeably and refer to a composition comprising at least one biocompatible skin augmentation material. It is to be understood that a skin augmentation composition according to the present invention is suitable for filling of skin, dermal layer, hypodermal layer or a combination thereof.

As used herein, the terms “biocompatible skin augmentation material”, “biocompatible soft tissue augmentation material”, “biocompatible agent” and “biocompatible material” are used interchangeably. As used herein, the term “biocompatible material” refers to a biocompatible skin augmentation material. According to some embodiments, the biocompatible material is an inorganic ceramic material, such as, but not limited to, hydroxyapatite. According to some embodiments, the biocompatible material is water-insoluble. According to some embodiments, the biocompatible material is a calcium phosphate ceramic material. As used herein, the terms “hydroxyapatite”, “hydroxylapatite”, “calcium hydroxyapatite” and “calcium hydroxylapatite” are interchangeable. According to some embodiments, hydroxyapatite as used herein further refers to a salt or derivative of hydroxyapatite.

In certain embodiments, the skin augmenting material is at least 95% crystalline. In certain embodiments, the skin augmenting material is at least 95% crystalline by XRD method. In certain embodiments, the skin augmenting material is at least 98% pure. In certain embodiments, the skin augmenting material has a specific weight of 0.45 g/cm³ to 0.65g/cm³. In certain embodiments, the skin augmenting material has a specific weight of 0.509g/cm³.

In certain embodiments, the hydroxyapatite is at least 95% crystalline. In certain embodiments, the hydroxyapatite is at least 95% crystalline by XRD method. In certain embodiments, the hydroxyapatite is at least 98% pure. In certain embodiments, the hydroxyapatite has a specific weight of 0.45 g/cm³ to 0.65g/cm³. In certain embodiments, the hydroxyapatite has a specific weight of 0.509g/cm³.

A non-limiting example of a skin augmentation composition comprising a biocompatible ceramic material is RADIESSE® manufactured by Merz Aesthetics, comprising calcium hydroxylapatite beads suspended in a gel carrier that consists primarily of water, glycerin and sodium carboxymethylcellulose.

According to some embodiments, a biocompatible material is biodegradable. According to some embodiments, a biocompatible material is capable of undergoing biodegradation not less than 1, 2, 3, 4 weeks following administration to a subject. According to some embodiments, a biocompatible material is capable of undergoing biodegradation not less than 1, 2, 3, 4, 5, 6, 7, 8, 9, 10, 11, 12 months following administration to a subject. According to some embodiments, a biocompatible material is capable of undergoing biodegradation not less than 0.5, 1, 2, 3 years following administration to a subject. According to some embodiments, a biocompatible material is capable of undergoing biodegradation not less than few months following administration to a subject. According to some embodiments, a biocompatible material is capable of undergoing biodegradation not less than 12 months following administration to a subject.

According to some embodiments, the biocompatible material is in the form of beads and/or particles. According to some embodiments, the biocompatible material comprises beads and/or particles having the same/different sizes. According to some embodiments, the biocompatible material is in the form of beads and/or particles of a size suitable for the size of the treated area. According to some embodiments, applicators which contain large beads of a biocompatible material are suitable for treating deep and/or large lines, wrinkles or folds.

According to some embodiments, the biocompatible material comprises beads and/or particles having a size of up to 5, 10, 20, 30, 40, 50, 60, 70, 80, 90 or 100 micrometers (μm). According to some embodiments, the biocompatible material comprises beads and/or particles having a size of 25-45 μm. According to some embodiments, the biocompatible material comprises beads and/or particles having a size of 10-50 μm. According to some embodiments, the biocompatible material comprises beads and/or particles having a size of 5-20 μm. According to certain embodiments, the biocompatible material comprises beads and/or particles having a size of about 40 μm. According to some embodiments, the biocompatible material particles are of about 10-100 μm, preferably of about 40 μm.

According to some embodiments, the skin augmentation composition comprises at least 1, 2, 3, 4, 5, 10, 15, 25, 30, 40, 50, 60, 70, 80, 90 or 95 percent biocompatible material. According to some embodiments, the skin augmentation composition comprises at least 30% biocompatible material.

According to some embodiments, the composition of the invention comprises at least one biocompatible filler, at least one biodegradable carrier and at least one additional skin augmentation material. According to some embodiments, the composition of the invention comprises hydroxyapatite and at least one biodegradable carrier. According to some embodiments, the composition of the invention comprises hydroxyapatite and polyethylene glycol. According to some embodiments, the composition of the invention comprises hydroxyapatite, polyethylene glycol and magnesium sulfate.

According to some embodiments, the biodegradable carrier is selected from the group consisting of: a salt, a biodegradable polymer and a combination thereof. According to some embodiments, the biodegradable carrier is a salt. According to some embodiments, the salt is a water-soluble salt. According to some embodiments, the salt is selected from the group consisting of: sodium sulfate, sodium chloride, magnesium sulfate, magnesium citrate, magnesium chloride and a combination thereof.

According to some embodiments, the biodegradable carrier is a biodegradable polymer. According to some embodiments, the biodegradable polymer is a polymer selected from the group consisting of: Polyethylene glycol (PEG), Polyglactin 910, Polyglecaprone 25, Polydioxanone, Lactomer 9-1, Glycomer 631, Polyglyconate and combinations thereof. According to some embodiments, the biodegradable carrier is magnesium sulfate and/or polyethylene glycol. According to some embodiments, PEG as used herein has a molecular weight between 10 and 50 kDa. According to some embodiments, a biodegradable carrier comprising PEG of 10-50 kDa has a thick paste consistency. According to some embodiments, the biodegradable carrier is Polyglactin 910 and/or magnesium sulfate.

According to some embodiments, the biodegradable carrier is degradable within 1, 2, 3, 4, 5, 6, 7, 8, 9, 10, 12 hours of inserting the microneedles into the skin of a subject. According to some embodiments, the biodegradable polymer is degradable within 1, 2, 3, 4, 5, 6, 7, 8, 9, 10, 12 hours of inserting the microneedles into the skin of a subject. According to some embodiments, the biodegradable carrier is degradable within 0.5, 1, 2, 3, 4, 5, 6, 7 days of inserting the microneedles into the skin of a subject. According to some embodiments, the biodegradable polymer is degradable within 0.5, 1, 2, 3, 4, 5, 6, 7 days of inserting the microneedles into the skin of a subject. According to some embodiments, the biodegradable carrier undergoes biodegradation within less than 7 days of inserting the microneedles into the skin of a subject, preferably less than 2 days, most preferably less than 1 day. According to some embodiments, rapid biodegradation of the biodegradable carrier within hours/days of introduction into the body of a subject results in uniform distribution of the biocompatible filler material and/or the skin augmentation material in the treated area, thus achieving uniform filing of the treated skin defect/deficiency. According to some embodiments, following insertion of the composition of the invention to the skin of the subject, the biodegradable carrier undergoes biodegradation and the biocompatible filler remains within the skin of a subject for at least several months, preferably up to a year, most preferably more than a year.

According to some embodiments, insertion of microneedles comprising the skin augmentation composition to the skin of a subject results in biodegradation of fast-degrading elements in the composition, thus resulting in release of the biocompatible filler into the treated area. According to some embodiments, the fast-degrading element is a biodegradable carrier such as, but not limited to, magnesium sulphate and/or polyethylene glycol. It is to be understood that, according to some embodiments, biodegradation of elements in the composition such as a biodegradable carrier assist in homogenous spreading of the biocompatible filler in the treated area. According to some embodiments, following biodegradation of fast-degrading elements, such as a biodegradable carrier, the biocompatible filler is transferred from the microneedle to the treated area. As used herein, fast-degrading elements refer to elements within the composition of the invention which undergo biodegradation within hours or up to 7 days from insertion of the microneedles of the invention into the skin of a subject. It is to be understood that a biocompatible filler is not a fast-degrading element of the composition of the invention. According to some embodiments, following administration of the applicator of the invention for a desired period of time, the applicator and the microneedles are removed from the subject, while at least part of the composition remains in the treated area.

According to some embodiments, the biodegradable carrier comprises water and/or glycerol and/or carboxymethylcellulose. According to some embodiments, the biodegradable carrier comprises water, glycerol and carboxymethylcellulose. According to some embodiments, the biodegradable carrier comprises carboxymethylcellulose.

According to some embodiments, the composition of the invention comprises a biocompatible filler material in the form of beads and/or particles surrounded by at least one biodegradable carrier. According to some embodiments, the composition of the invention comprises a biocompatible filler material in the form of beads and/or particles surrounded by at least one biodegradable polymer. According to some embodiments, the composition of the invention comprises a biocompatible filler material in the form of beads and/or particles surrounded by at least one salt. According to some embodiments, the composition of the invention comprises hydroxyapatite in the form of beads and/or particles surrounded by at least one biodegradable carrier.

According to some embodiments, beads or particles of a biocompatible filler material such as, but not limited to, hydroxyapatite, surrounded by a biodegradable carrier, homogeneously spread in the treated area upon degradation of the biodegradable carrier by dissolution, enzymatic activity and the like.

According to some embodiments, adding a biodegradable polymer to the composition of the invention results in a composition having a gel, a paste or a solid like consistency. According to some embodiments, a gel, a paste or a solid like composition are configured to be easily inserted into and kept in the middle part of the microneedles of the invention. According to some embodiments, addition of a salt to the composition of the invention assists in uniform dispersion of the biocompatible filler within the composition. According to some embodiments, an addition of a material that attracts water or water solution (like glycerin, or salt, or polyethylene glycol (PEG), and other such materials or compound) to the composition of the invention results in water diffusion into the composition, thus assisting in uniform dispersion of the biocompatible filler within the composition and/or within the treated area.

As used herein, the terms “skin augmentation material” and “filler” refer to agents and compositions useful for augmentation of skin defects. According to some embodiments, a skin augmentation material is a dermal and/or hypodermal filler. Suitable skin augmentation materials according to the invention include, but are not limited to, proteins, polysaccharides, lipids, synthetic polymers and combinations thereof. According to some embodiments, a skin augmentation material according to the invention is any material known in the art which is suitable for filling undesired fold, wrinkle, depressed scar or line in a skin of a subject. According to some embodiments, a skin augmentation material according to the invention is any skin augmentation material which is configured to be delivered using microneedles. According to some embodiments, a biocompatible filler material is a skin augmentation material. According to certain embodiments, a skin augmentation material refers to a biocompatible, inert material. The term “Inert material” as used herein refers to a non-antigenic, non-carcinogenic, non-teratogenic, and non-migratory augmentation material.

According to some embodiments, skin augmentation materials include allogeneic products, xenogeneic products and synthetically derived products.

According to some embodiments, the composition of the invention further comprises at least one skin augmentation material selected from the group consisting of: a biodegradable natural substance, a biodegradable synthetic polymer, a non-biodegradable synthetic polymer, a non-biodegradable natural substance and combinations thereof.

According to some embodiments, a biodegradable natural substance is selected for example from the group consisting of: bovine collagen, porcine collagen, recombinant collagen, human collagen, gelatin, hyaluronic acid, hyaluronic acid derivative, dried acellular particulate dermal matrix, allogeneic fat and combinations thereof.

According to some embodiments, a biodegradable synthetic polymer is selected for example from the group consisting of: poly-L-lactic acid, polyethylene oxide, carboxymethylcellulose and combinations thereof.

According to some embodiments, a non-biodegradable synthetic polymers is selected for example from the group consisting of: polymethyl methacrylate (PMMA), polymethyl methacrylate beads, silicones, silicone rubber, expanded polytetrafluoroethylene (ePTFE), polyacrylamide, polyalkylimide and combinations thereof.

According to some embodiments, the skin augmentation composition comprises at least one biocompatible filler material, a biodegradable carrier and at least one type of skin augmentation material selected from the group consisting of: a biodegradable natural substance, a biodegradable synthetic polymer, a non-biodegradable synthetic polymer and combinations thereof.

According to some embodiments, the skin augmentation composition comprises hydroxyapatite and at least one type of skin augmentation material other than hydroxyapatite. According to some embodiments, the skin augmentation composition comprises hydroxyapatite and at least one type of soft-tissue augmentation material selected from the group consisting of: a biodegradable natural substance, a biodegradable synthetic polymer, a non-biodegradable synthetic polymer, a non-biodegradable natural substance and combinations thereof. According to some embodiments, the skin augmentation composition comprises hydroxyapatite and at least one type of soft-tissue augmentation material selected from the group consisting of: a biodegradable natural substance, a biodegradable synthetic polymer, a non-biodegradable synthetic polymer and combinations thereof.

According to some embodiments, the composition of the invention comprises less than 50% weight percent water-soluble skin augmentation materials such as, but not limited to, collagen, hyaluronic acid and gelatine.

Skin augmentation materials, which are to be comprised in the composition of the invention are effective dermal fillers approved by the U.S. Food and Drug administration, including but not limited to fillers comprising structural proteins, polysaccharides or synthetic polymers. Exemplary embodiments of skin augmentation materials that are to be used include collagen, such as reconstituted bovine collagen products including, but not limited to, ZYDERM I®, ZYDERM II® and ZYPLAST® (Collagen Corporation); natural human collagen COSMODERM™ and COSMOPLAST™ (INAMED); and endogenous collagen from the subject, AUTOLOGEN® produced by Collagenesis. According to some embodiments, additional examples of dermal fillers can be selected from those comprising hyaluronic acid, including but not limited to, such products as HYLAFORM® gel manufactured by INAMED and Genzyme Corporations, derived from the rooster combs of domestic fowl; and RESTYLANE® manufactured by Medicis, a hyaluronic acid derivative derived from streptococcal bacterial fermentation. Hyaluronic acid according to the present invention includes both non-cross-liked and/or cross-linked hyaluronic acid derivatives as are well known in the art. “Hyaluronic acid”, according to the present invention, includes solid and semi solid forms of Hyaluronic acid. According to some embodiments, collagen according to the invention is selected from the group consisting of: allogeneic collagen, xenogeneic collagen and a combination thereof. According to other embodiments, a skin augmentation material is human cadaveric dermis cultivated from a cadaver.

According to some embodiments, the composition of the invention further comprises a biologically active agent. According to some embodiments, the biologically active agent is selected from the group consisting of: an enzyme, a drug, a toxin and a combination thereof. According to some embodiments, the composition of the invention is devoid of any biologically active agents.

According to some embodiments the enzyme is collagenase for treating scars or keloids, hyaluronidase to treat Hyaluronic acid excess, or elastase for skin expansion.

According to some embodiments, the drug is an analgesic. According to some embodiments, when the applicator of the invention is used to deliver skin augmentation composition subcutaneously, at least one analgesic is co-delivered by the applicator of the invention together with the skin augmentation composition. According to some embodiments, the skin augmentation composition of the invention further comprises an analgesic. According to some embodiments, the methods of the invention further comprise administration of an analgesic. According to some embodiments, every analgesic known in the art can be used with the present invention, such as, but not limited to: lidocaine, paracetamol, non-steroidal anti-inflammatory drug (NSAID), COX-2 inhibitor, opiates or morphinomimetics. According to some embodiments, an analgesic which can be used with the present invention is lidocaine.

According to some embodiments, the drug is a drug known in the art to assist in filling undesired lines, wrinkles, folds and the like. According to some embodiments, examples of drugs which are suitable to be comprised in the composition of the invention include, but are not limited to, anti-psoriasis drugs, muscle relaxants and a combination thereof.

According to some embodiments, the drug is a drug for treatment or prevention of pathological scarring. According to some embodiments, the drug for treatment of pathological scarring is a corticosteroid. According to some embodiments, the corticosteroid is any corticosteroid known in the art for treatment of pathological scarring, such as, but not limited to triamcinolone.

According to some embodiments, the toxin is botulinum toxin. According to some embodiments, the composition of the invention comprises botulinum toxin. According to some embodiments, the composition of the invention comprises botulinum toxin type A, human albumin and sodium chloride. According to some embodiments, the applicator of the invention comprises botulinum toxin.

According to some embodiments, the skin augmentation composition of the invention further comprises a medical pigment. According to some embodiments, the microneedles of the invention further comprise a medical pigment. As used herein, the term “medical pigment” refers to a color material suitable for insertion into the skin of a subject. According to some embodiments, medical pigments have a regulatory approval for insertion into a skin of a subject. According to some embodiments, medical pigments are pigments known in the art to be suitable for micro-pigmentation treatments. In non-limiting examples, medical pigments suitable for use according to the present invention include, but are not limited to, pigments such as BIOCHROMADERM® (Biotic Phocea) or the Signature Series (Micro-Pigmentation Centre, Inc.). According to some embodiments, possible medical pigments for use with the applicator of the present invention can be selected from: pigments for scar camouflage, areola reconstruction, lip remodeling and any combination thereof.

According to some embodiments, a microneedle comprising a medical pigment is suitable for micro-pigmentation treatments. According to some embodiment, micro-pigmentation treatments are selected from the group consisting of: concealment of scars, concealment and/or blurring of skin pigmentation, nipple areola construction and/or augmentation, correction of freckles, lip coloring, eyebrow coloring and a combination thereof. According to some embodiments, the microneedles of the invention comprise a composition comprising a medical pigment. According to some embodiments, the applicator of the invention further comprises microneedles comprising a medical pigment without a biocompatible filler or a skin augmentation composition.

As used herein, the terms “subject”, “a subject in need thereof” and “a patient in need thereof” are used interchangeably and refer, according to some embodiments, to a subject in need of skin or sub cutis augmentation or a combination thereof. According to some embodiments, the subject is a subject having undesired lines, wrinkles, and folds such as, but not limited to, elderly people. According to other embodiments, the subject is a subject having a scar in need of augmentation or filling. In a non-limiting example, a subject is a subject having facial wrinkles which he or she would like to have filled for a younger, healthier and fuller looking facial skin. Of note, a subject may have normal looking skin and wish to use the applicator/method of the invention in order achieve an appearance of fuller skin at a desired area, such as, but not limited to, the cheeks and lips.

The term “substantially” as used herein refers, according to some embodiments, to the complete or nearly complete extent or degree of an action, characteristic, property, state, structure, item, or result. For example, a composition which is “substantially consists of A and B” would mean that the composition is either completely made of A and B or nearly completely made of A and B, taking into account minute impurities. The exact allowable degree of deviation from absolute completeness depends in some cases on the specific context. However, generally speaking the nearness of completion will be so as to have the same overall result as if absolute and total completion were obtained.

The use of “substantially” is equally applicable when used in a negative connotation to refer, according to some embodiments, to the complete or near complete lack of an action, characteristic, property, state, structure, item, or result. For example, a composition that is “substantially free of A” would either completely lack A, or so nearly completely lack A that the effect would be the same as if it completely lacked A. In other words, a composition that is “substantially free/devoid of” an ingredient or element may still actually contain such item as long as there is no measurable effect thereof.

As used herein, the term “about” is used to provide flexibility to a provided numerical value, by providing that a given value may be “a little above” or “a little below” the provided number. As used herein, the term “about” further refers, according to some embodiments, to ±10%, preferably +5%, and most preferably ±1% of the mentioned numerical value.

As used herein, the terms “sub cutaneous” and “sub cutis” are used interchangeably. It is to be understood that the applicator and/or the microneedles of the invention are configured for administration of a skin augmentation composition to skin or to sub-cutaneous layers or to a combination thereof. It is to be understood that the methods of the invention provide augmentation or filling of skin or sub cutaneous layers or a combination thereof.

According to some embodiments, the present invention provides a use of the applicator of the invention for augmentation of skin in a subject in need thereof. According to some embodiments, the present invention provides a use of the applicator of the invention for the filling of an undesired fold, wrinkle, line or depressed area in the skin of a subject in need thereof.

As used herein, the term “biocompatible filler soft-tissue augmentation material”, “biocompatible filler material” and “biocompatible filler” are used interchangeably.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A device comprising:
a sleeve body defining an internal bore and terminating at a distal opening;
a hollow microneedle defining an internal lumen and terminating at a distal pointed tip having an outlet, wherein said microneedle is configured to be reciprocally displaceable within said internal bore such that said pointed tip extends and retracts through said distal opening;
an aperture in a sidewall of said microneedle which provides communication between a reservoir storing a specified composition and said internal lumen, to allow for ingress of a quantity of said specified composition from said reservoir into said internal lumen; and
a plunger having a shaft which is operable for advancing a seal along said internal lumen to collect said quantity of said specified composition communicated into said internal lumen and to deliver at least a portion of said quantity of said specified composition through said outlet.

2. The device of claim 1, wherein said reservoir is attached at a proximal end of said body sleeve.

3. (canceled)

4. The device of claim 1, wherein said pointed tip is configured to puncture a skin of a subject at an injection site.

5. The device of claim 4, wherein said distal opening is configured to rest against an external surface of said skin of said subject at said injection site, and wherein said microneedle is configured to extend though said distal opening a predetermined distance into a dermal layer or hypodermal layer of said subject, to deliver said at least a portion of said quantity of said specified composition at a desired depth location within said dermal or hypodermal layers.

6. The device of claim 1, wherein said microneedle is made of a material selected from a group consisting of: metal, plastic, a ceramic material, a silicone material, and any combination thereof.

7. The device of claim 1, wherein said microneedle has a cross-sectional shape selected from the group consisting of: circular, rectangular, oval, triangular, and polygonal.

8. The device of claim 1, wherein said pointed tip comprises a tip having an angle of convergence of between 10-60 degrees.

9. The device of claim 1, wherein said specified composition is a skin augmentation composition.

10. The device of claim 1, wherein said specified composition has a consistency that is liquid, viscous, semisolid, or solid.

11. (canceled)

12. (canceled)

13. The device of claim 1, further comprising one or more sensors configured to perform imaging or depth measurement of a local skin structure at said injection site, wherein said one or more sensors are selected from the group consisting of: an imaging device, an ultrasound sensor, an optical transducer, and a pressure gage force transducer.

14. (canceled)

15. (canceled)

16. A method comprising:
providing a device comprising:
a sleeve body defining an internal bore and terminating at a distal opening,
a hollow microneedle defining an internal lumen and terminating at a distal pointed tip having an outlet, wherein said microneedle is configured to be reciprocally displaceable within said internal bore such that said pointed tip extends and retracts through said distal opening,
an aperture in a sidewall of said microneedle which provides communication between a reservoir storing a specified composition and said internal lumen, to allow for ingress of a quantity of a specified composition from said reservoir into said internal lumen, and
a plunger having a shaft which is operable for advancing a seal along said internal lumen to collect said quantity of said specified composition communicated into said internal lumen and to deliver at least a portion of said quantity of said specified composition through said outlet;
locating said device at an injection site about a skin of a subject;
displacing said microneedle within said internal bore such that said pointed tip extends a predetermined distance through said distal opening into a dermal layer or hypodermal layer of said skin of said subject; and
operating said shaft to advance said seal along said internal lumen to deliver said at least a portion of said quantity of said specified composition through said outlet, into said dermal or hypodermal layers.

17. The method of claim 16, wherein said reservoir is attached at a proximal end of said body sleeve.

18. (canceled)

19. The method of claim 16, wherein said pointed tip is configured to puncture said skin of said subject at said injection site.

20. The method of claim 19, wherein said distal opening is configured to rest against an external surface of said skin of said subject at said injection site, and wherein said predetermined distance defines a desired depth location within said dermal or hypodermal layers for delivering said at least a portion of said quantity of said specified composition.

21. The method of claim 16, wherein said microneedle is made of a material selected from a group consisting of: metal, plastic, a ceramic material, a silicone material, and any combination thereof.

22. The method of claim 16, wherein said microneedle has a cross-sectional shape selected from the group consisting of: circular, rectangular, oval, triangular, and polygonal.

23. The method of claim 16, wherein said pointed tip comprises a tip having an angle of convergence of between 10-60 degrees.

24. The method of claim 16, wherein said specified composition is a skin augmentation composition.

25. The method of claim 16, wherein said specified composition has a consistency that is liquid, viscous, semisolid, or solid.

26. (canceled)

27. (canceled)

28. The method of claim 16, wherein said device further comprises one or more sensors configured to perform imaging or depth measurement of a local skin structure at said injection site, wherein said one or more sensors are selected from the group consisting of: an imaging device, an ultrasound sensor, an optical transducer, and a pressure gage force transducer.

29. (canceled)

30. (canceled)
